(19) United States
(12) Patent Application Publication
(10) Pub. No.: US 2004/0193054 A1
(43) Pub. Date: Sep. 30, 2004

(54) Hand-held ophthalmic device

(76) Inventors: Paul D. Leblanc, Greenbush, MA (US); Niels E. Andersen, Toronto (CA)

Correspondence Address: FISH & RICHARDSON PC, 225 FRANKLIN ST, BOSTON, MA, 02110, US

(21) Appl. No.: 10/371,720

(22) Filed: Feb. 19, 2003

(51) Int. Cl. A61B 8/00

(52) U.S. Cl. 600/452

(57) ABSTRACT

A hand-held pachymeter includes an ultrasonic probe capable of emitting ultrasonic pulses and capable of receiving echo signals of the ultrasonic pulses. The echo signals may include an echo signal from an anterior portion of a cornea of an eye and an echo signal from a posterior portion of the cornea. The hand-held pachymeter also includes a hand-held computing device and an expansion module that interfaces the probe to the hand-held computing device. The expansion module includes circuitry to produce digitized signals that correspond to the echo signals, and a memory which stores computer instructions to determine a thickness of the cornea based on the digitized signals. The computer instructions are executed by the hand-held computing device.

### BACKGROUND

This invention relates to a hand-held ophthalmic device, ultrasonic or otherwise, such as a pachymeter.

An ophthalmic pachymeter (or simply “pachymeter”) is a diagnostic measuring instrument that uses ultrasound technology to measure the thickness of the cornea of an eye. In operation, the pachymeter is placed in the vicinity of (e.g., in contact with) a patient's eye. The pachymeter emits an ultrasonic pulse, which reflects off of portions of the patient's cornea, resulting in echo signals. The pachymeter processes these echo signals to determine the thickness of the cornea.

Heretofore, hand-held ophthalmic devices, such as pachymeters were expensive and difficult to produce, in part, due to custom-designed circuits and electronics contained therein.

### SUMMARY

The present invention addresses drawbacks associated with conventional ophthalmic devices by using a commercially-available hand-held computing device, such as a Handspring™ Visor™, as the computing and human interface portions of the ophthalmic device. As described herein, a hardware module that “plugs-in” to the hand-held computing device transforms the hand-held computing device into a hand-held ophthalmic device.

In general, in one aspect, the invention is directed to an ultrasonic device that includes a probe to receive an ultrasonic signal from an eye, a hand-held computing device, and an expansion module which interfaces the probe to the hand-held computing device. The expansion module includes circuitry to process the ultrasonic signal and executable instructions to obtain a characteristic of the eye based on the ultrasonic signal. This aspect may include one or more of the following features.

The probe may be a piezoelectric transducer. The piezoelectric transducer may generate a pulse which propagates to a structure in the eye. The piezoelectric transducer may receive an echo of the pulse off of the structure. The echo may be the ultrasonic signal. The structure may be at least one of a cornea of the eye, a lens of the eye, and a retina of the eye.

The hand-held computing device may include a user interface. The user interface may display data that corresponds to the ultrasonic signal and may be used for inputting commands. The circuitry may include a memory that stores the executable instructions, circuits to produce a digitized ultrasonic signal from the ultrasonic signal, and processing logic which performs processing of the digitized ultrasonic signal.

Processing the digitized ultrasonic signal may include filtering the digitized ultrasonic signal to reduce noise; combining the digitized ultrasonic signal with other digital signals to produce an averaged signal; performing a correlation operation on the digitized ultrasonic signal; performing peak detection to locate a feature of the digitized ultrasonic signal; and/or performing an interpolation using the digitized ultrasonic signal. The expansion module may include an expansion slot interface to the hand-held computing device.

In general, in another aspect, the invention is directed to a hand-held pachymeter that includes an ultrasonic probe capable of emitting ultrasonic pulses and capable of receiving echo signals of the ultrasonic pulses. The echo signals may include an echo signal from an anterior portion of a cornea of an eye and an echo signal from a posterior portion of the cornea. The hand-held pachymeter also includes a hand-held computing device and an expansion module that interfaces the probe to the hand-held computing device. The expansion module includes circuitry to produce digitized signals that correspond to the echo signals, and a memory which stores computer instructions to determine a thickness of the cornea based on the digitized signals. The computer instructions are executed by the hand-held computing device.

This aspect may include one or more of the following features. The ultrasonic probe may be a piezoelectric transducer. The circuitry may include an amplifier to amplify the echo signals and a digitizer to digitize amplified echo signals to produce the digitized signals. The circuitry may filter the digitized signals to reduce noise, combine the digitized signals with other digital signals to produce an averaged digitized signal, perform a correlation operation on a digitized signal, perform peak detection to locate a feature of a digitized signal, and/or perform an interpolation using the digitized signals.

In general, in another aspect, the invention is directed to a hand-held ultrasonic device, which includes receiving means for receiving an ultrasonic signal from a structure of an eye, and processing means for processing the ultrasonic signal to determine a characteristic of the structure. The processing means includes a hand-held computing device for receiving data from the receiving means that is used in determining the characteristic of the structure. This aspect of the invention may include one or more of the following features.

The receiving means may be an ultrasonic probe capable of emitting ultrasonic pulses and capable of receiving ultrasonic echo signals of the ultrasonic pulses. The ultrasonic echo signals may include an echo signal from an anterior portion of a cornea of an eye and an echo signal from a posterior portion of the cornea. The processing means may include circuitry to produce digitized signals that correspond to the ultrasonic echo signals and a processor to determine a thickness of the cornea based on the digitized signals. The receiving means may include a transducer that generates a pulse that propagates to a structure in the eye and receives an echo of the pulse off of the structure. The echo may be the ultrasonic signal. The structure may include at least one of a cornea of the eye, a lens of the eye, and a retina of the eye.

In general, in another aspect, the invention is directed to a medical device. The medical device includes a probe to receive a signal from an eye, a hand-held computing device, and an expansion module which interfaces the probe to the hand-held computing device. The expansion module includes circuitry to process the signal and to provide a processed version of the signal to the hand-held computing device. This aspect of the invention may include one or more of the following features.

The probe may be an ultrasonic probe, a strain gauge, a pressure transducer, and/or a force gauge. The characteristic of the eye may include pressure in the eye.

If the probe is ultrasonic, the probe may be capable of emitting ultrasonic pulses and may be capable of receiving ultrasonic echo signals of the ultrasonic pulses. The signal may include at least one of an ultrasonic echo signal from an anterior portion of a cornea of an eye and an ultrasonic echo signal from a posterior portion of the cornea. The circuitry may include circuitry to produce digitized signals that correspond to the ultrasonic echo signals, and processing logic to determine a thickness of the cornea based on the digitized signals.

Other features and advantages of the invention will become apparent from the following description, including the claims and drawings.

### DESCRIPTION OF THE DRAWINGS

FIG. 1 is a block diagram of a hand-held ophthalmic device showing components thereof.

FIG. 2 is a diagram showing the hand-held ophthalmic device in use on a patient's eye.

FIG. 3 is a block diagram of circuitry contained in an expansion module in the hand-held ophthalmic device.

FIG. 4 is a graph showing ultrasonic pulses and echoes used with the hand-held ophthalmic device.

FIG. 5 is a front view of the hand-held ophthalmic device and its graphical user interface (GUI).

FIGS. 6 and 7 show graphical displays provided on the GUI of the hand-held ophthalmic device.

Like reference numerals in different figures indicate like elements.

### DESCRIPTION

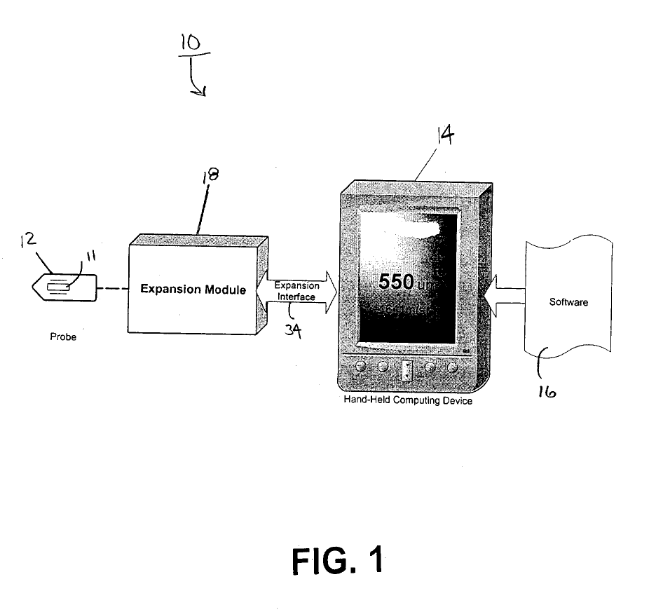

FIG. 1 shows a block diagram of a hand-held ophthalmic device. The device shown in FIG. 1 is an ultrasonic pachymeter (hereinafter “pachymeter”) 10; however, as described below, the block diagram of FIG. 1 may represent different types of hand-held medical devices.

Briefly, pachymeter 10 includes a probe 12 to receive ultrasonic signals from a patient's eye, a hand-held computing device 14 that runs software 16, and an expansion module 18 that interfaces probe 12 to hand-held computing device 14. Expansion module 18 includes circuitry to process the ultrasonic signal and executable instructions to obtain a characteristic of the eye, such as corneal thickness, based on the ultrasonic signal.

In this embodiment, probe 12 is an ultrasonic probe that contains a piezoelectric transducer 11 for generating and receiving the ultrasonic signals (in this case, ultrasonic pulses). The ultrasonic pulses used in this embodiment have a central frequency of about 20 MHz (Megahertz), although any appropriate frequency signals may be used.

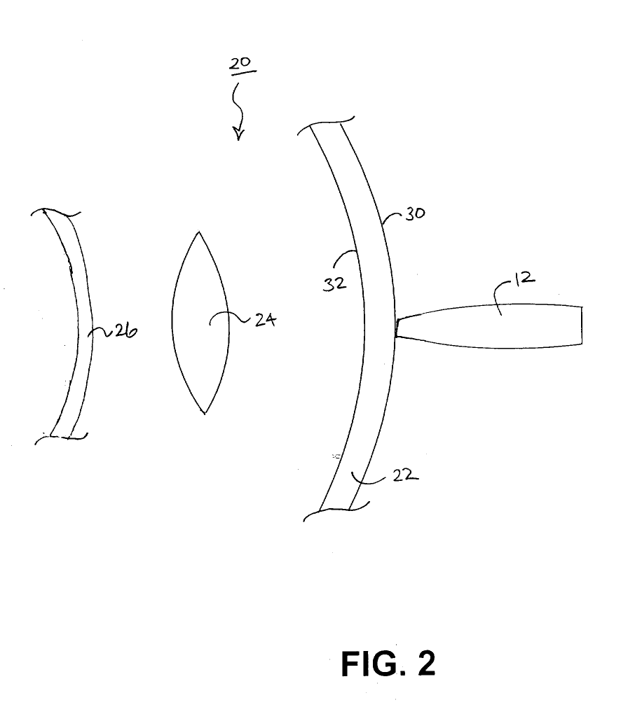

Referring to FIG. 2, probe 12 emits ultrasonic pulses in the vicinity of (e.g., in contact with) a patient's eye 20. The contact surface between the eye and the probe is about ⅛ inches in this embodiment. As shown in FIG. 2, eye 20 includes cornea 22, lens 24, and retina 26. Cornea 22 has anterior portion 30, which is closest to probe 12, and posterior portion 32, which is farthest from probe 12. Lens 24 and retina 26 contain corresponding anterior and posterior portions, as also shown in FIG. 2.

The ultrasonic pulses transmitted by probe 12 reflect off of structures (e.g., the cornea, lens and retina) in the patient's eye. Reflections occur off of both the anterior and posterior portions of such structures. Probe 12 receives ultrasonic echo signals (or simply “echoes”) resulting from these reflections and transmits the echoes to expansion module 18, which is described below.

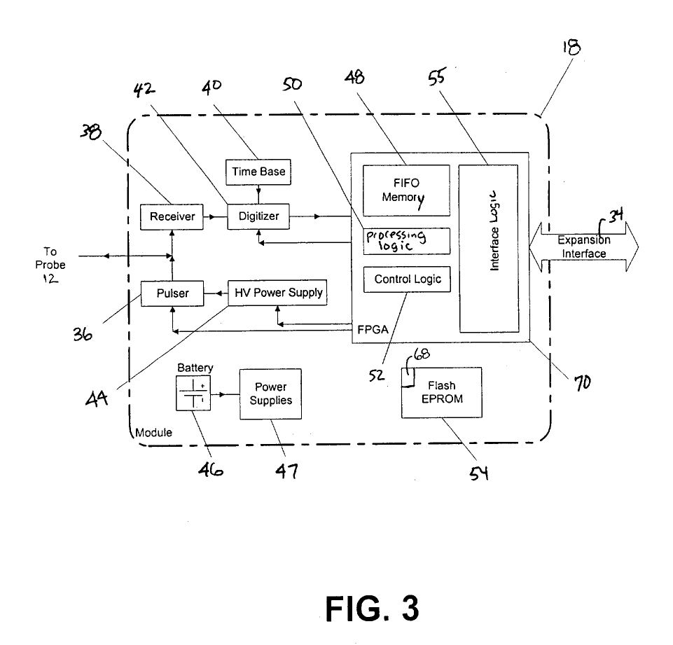

Expansion module 18 processes the echoes received from the patient's eye and provides data for use by hand-held computing device 14 in determining characteristics of the eye. In a pachymeter, the characteristic is corneal thickness; however, other characteristics may be determined depending upon the source of the echoes. In this embodiment, hand-held computing device 14 is a Handspring™ Visor™. Expansion module 18 connects to hand-held computing device 14 via a standardized electro-mechanical expansion interface 34 (see also FIG. 3), such as the standard Handspring™ Springboard™ expansion slot. Hand-held computing device 14 runs the PalmOS™ operating system; however other operating systems may be used. Software 16 running on hand-held computing device 14 provides a user interface, operating system interface, and interfaces to components (hardware) on expansion module 18.

Referring to FIG. 3, expansion module 18 includes the following circuitry: pulser 36, receiver 38, time base 40, digitizer 42, high voltage (HV) power supply 44, battery 46, power supplies 47, first-in first-out (FIFO) memory 48, processing logic 50, control logic 52, flash electrically-erasable programmable read-only memory (EPROM) 54, interface logic 55, and interface 34.

HV power supply 44 generates, from lower operating voltages (e.g., 2 V (Volts) or 3 V) on expansion module 18, a high voltage of sufficient magnitude and capacity to excite piezoelectric transducer 11. To this end, HV power supply 44 may contain a capacitor (e.g., a 1000 picofarad (pF)) capacitor that is charged to the high voltage. The high voltage is 150 V; however, other voltages may be used. HV power supply 44 may charge the capacitor from the voltage of battery 46 via power supplies 47.

Pulser 36 generates impulses from the high voltage provided by HV power supply 44 and applies these impulses to piezoelectric transducer 11. This embodiment uses a negative impulse having a substantially sharp decline, followed by a gradual rise up to the baseline (e.g., zero); although other impulses may be used. The pulse width may be 50 nanoseconds (ns). Pulser 36 may be implemented using a transistor switch that is gated to generate the impulses.

Piezoelectric transducer 11 generates ultrasonic pulses from the impulse(s) provided by pulser 36. These ultrasonic pulses propagate through the patient's eye when probe 12 is brought into contact with the eye. The ultrasonic pulses reflect off of structures in the eye, resulting in echoes (noted above) that are received by probe 12. In the case of a pachymeter, the ultrasonic pulses reflect off of anterior and posterior portions of the cornea, resulting in corresponding echo signals.

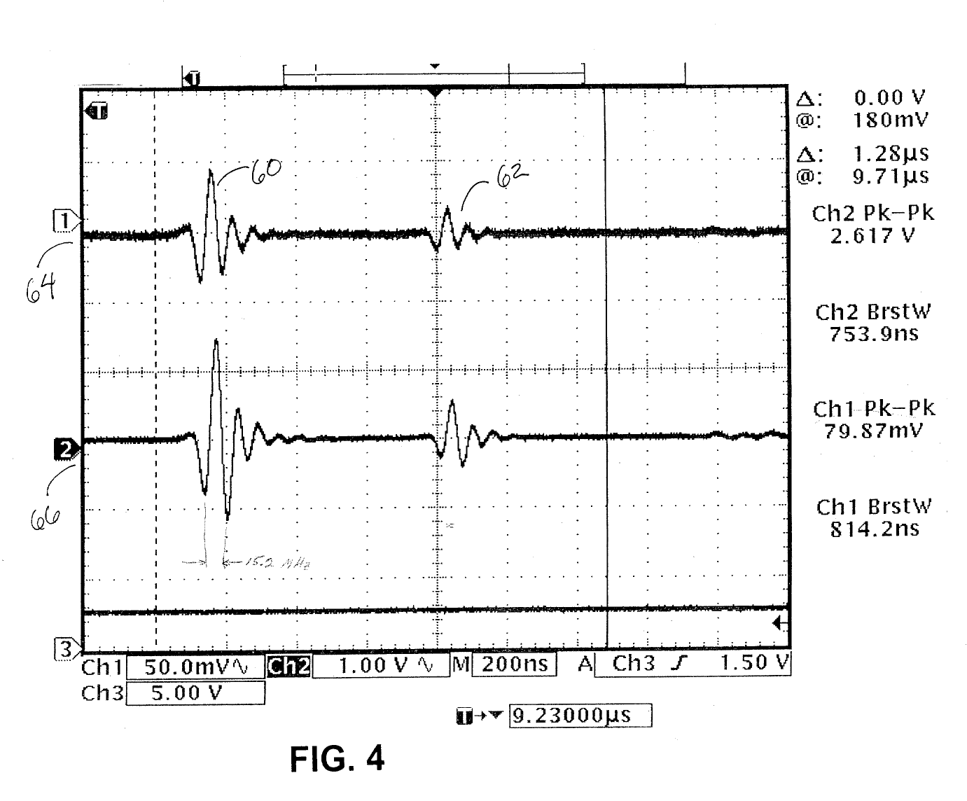

FIG. 4 shows an example of a pulse 60 and its echo 62. Specifically, FIG. 4 shows an oscilloscope display of an ultrasound pulse 60 having a central frequency of about 15 MHz and its echo 62 in water, which occurs roughly 700 ns later. The top waveform 64 shows the original pulse and echo. The bottom waveform 66 shows amplified versions of the pulse and echo signals shown in the top waveform.

Receiver 38 receives the echo signals from probe 12 and amplifies the echo signals to a level that is compatible with input requirements of digitizer 42. Receiver 38 may include a voltage limiting circuit (not shown) that allows receiver 38 to withstand high voltage impulses used to excite piezoelectric transducer 11.

Digitizer 42 is an analog-to-digital converter. Digitizer 42 converts analog ultrasonic pulses from the output of receiver 38 to digital (digitized) ultrasonic pulses at a sample rate determined by time base 40 (which may be a 100 MHz clock). Time Base 40 provides a precise timing signal for the digitizer 42 such that the time between subsequent input samples is known. The analog-to-digital converter used to implement digitizer 42 is of sufficient resolution and performance to accurately capture the dynamic range and frequency content of the echoes.

Resulting digitized echo signals are captured and stored in FIFO memory 48. One or more FIFO memories may be used. Processing logic 50 retrieves these signals from FIFO memory 48 and processes them as described below.

Processing logic 50 may be implemented using programmable logic (e.g., a field programmable gate array (FPGA)). However, the invention is not limited as such. For example, processing logic 50 may be implemented using non-programmable hardware (e.g., discrete logic circuits), a digital signal processor (DSP), a microprocessor/central processing unit (CPU), or any combination of these.

In this embodiment, processing logic 50 operates in conjunction with a processor (e.g., microprocessor, DSP, programmable logic, etc.) on hand-held computing device 14. In more detail, processing logic 50 may perform various type of processing on digitized echo signals. Thereafter, the processor on hand-held computing device 15 may determine the thickness of the patient's cornea (or other characteristic of the patient's eye) using the digitized echo signals that were processed by processing logic 50.

Examples of signal processing functions that may be performed by processing logic 50 include, but are not limited to, filtering the digitized echo signals, combining the digitized echo signals with other digitized echo signals to produce an averaged signal, performing a correlation operation (e.g., auto-correlation) on the digitized echo signal, performing peak detection to locate a feature of the digitized echo signal, and performing an interpolation using the digitized echo signal.

In more detail, processing logic 50 may perform filtering, such as bandpass filtering, to reduce noise in the digitized echo signals. Processing logic 50 may average digitized echo signals over a period of time and provide resulting averages for use in determining corneal thickness. Processing logic 50 may perform auto-correlation on the digitized echo signals or processing logic 50 may correlate the digitized echo signals to other digital signals. Processing logic 50 may perform peak detection processing on the digitized echo signals in order to identify similar points in each waveform. These points may be used in determining the time difference between pulses. Processing logic 50 may perform a spectral analysis (e.g., an interpolation using fast Fourier transforms (FFTs)) to locate digitized echo pulses.

Processing logic 50 may store the processed digitized echo signals back in FIFO memory 48 for subsequent retrieval and processing by the processor on hand-held computing device 14 (hereinafter, “the processor”). Alternatively, processing logic 50 may provide the processed digitized echo signals to the processor.

In the case of a pachymeter, the processor determines the thickness of the patient's cornea based on the echo signals received from the anterior and posterior portions of the cornea, which were digitized by digitizer 42 and processed by processing logic 50. The processor determines the thickness by identifying echo signals from the anterior and posterior portions of the cornea and determining the time difference (e.g., the number of clock cycles) between receipt of the anterior and posterior echo signals.

Knowing the speed of sound through the eye tissue being measured enables the processor (e.g., on hand-held computing device 14) to identify the echo signals and to calculate the thickness of the cornea from the time difference between the echo signals. The speed of sound through a human cornea is 1641 meters-per-second (m/s). This value may be used as a default; however, other values can be used, depending upon the structure being examined and the type of patient. These other values may be input by a user via a user interface on hand-held computing device 14 and/or downloaded from another source.

The processor on hand-held computing device 14 may execute software 68 (i.e., machine-executable instructions) to perform the foregoing signal processing functions, including determining corneal thickness. Software 68 may be stored in flash EPROM 54 on expansion module 18, as shown, and executed out of random access memory (RAM), which may be on hand-held computing device 14 or expansion module 18. Alternatively, the software, or a portion thereof, may be stored on hand-held computing device 14.

Control logic 52 is comprised of circuitry that controls and monitors timing and operation of the other circuitry resident on expansion module 18. For example, control logic 52 may command pulser 36 to produce pulses, charge HV power supply 44, and the like.

Interface logic 55 contains I/O (input/output) registers and circuitry specifically designed to interface to a particular type of expansion interface. In this embodiment, the circuitry interfaces to a Handspring™ Springboard™ expansion slot. The circuitry provides a way for software 16 running on hand-held computing device 14 to interact with, control, and respond to hardware and software contained in expansion module 18.

One or more of control logic 52, interface logic 55, processing logic 50, and FIFO memory 48 may be implemented using programmable logic, such as FPGA 70. In this embodiment, a RAM-based FPGA is used. However, discrete components such as those noted above may be used in addition to, or instead of, programmable logic.

As noted above, flash EPROM 54 contains software 68 for performing the signal processing functions described above, such as determining corneal thickness or making other measurements. Flash EPROM 54 may also contain configuration data for programming FPGA 70 or other data for use by hand-held computing device 14 or expansion module 18. The contents of flash EPROM 54 may be updated via hand-held computing device 14, thereby allowing for relatively easy distribution and installation of software or firmware updates on expansion module 18.

Power supply circuitry in power supplies 47 generates appropriate voltages to power all circuits from an available power supply, in this case, battery 46. Expansion module 18 may be powered from one or more separate batteries. Alternatively, power for expansion module may be provided by hand-held computing device 14 via expansion interface 34. Power supply(ies) on expansion module 18 may be recharged directly or via existing power supplies on hand-held computing device 14.

Interface 34 may be a standard electrical, mechanical, and software interface between hand-held computing device 14 and expansion module 18. As noted above, the Handspring™ Springboard™ expansion slot is an example of an expansion module interface. Another example of an expansion interface is the PCMCIA (Peripheral Component Microchannel Interconnect Architecture) expansion slot found on many hand-held computing devices.

Although use of a Handspring™ Visor™ is described herein, any type of hand-held computing device may be used. Examples of such hand-held computing devices include, but are not limited to, the Compaq® iPAQ®, the Hewlett Packard® Jornada®, and the PalmPilot®. An appropriate operating system is loaded onto the hand-held computing device.

Software 16 on hand-held computing device 14 provides, among other things, a user interface, an operating system interface, and an expansion module interface. Software 16 provides an efficient and intuitive interface for a user of pachymeter 10 to control its operation and to receive information therefrom. The user interface includes various graphical user interfaces (GUIs), including screens and menus presented on a liquid crystal display (LCD). Alternatively, these features may be provided by software 68 stored on flash EPROM 54.

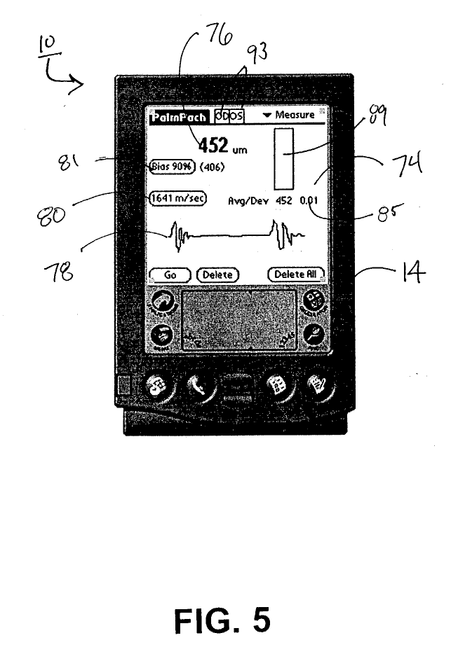

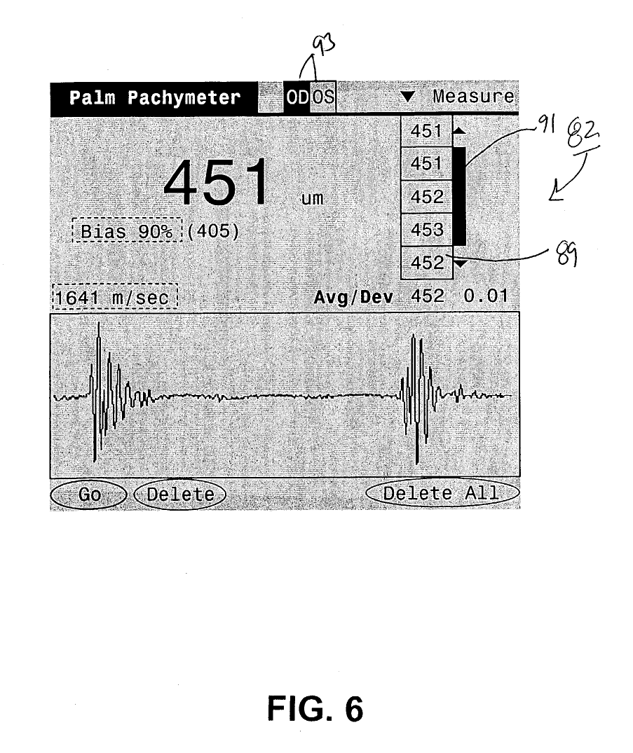

FIG. 5 shows an example of a display 74 provided on the user interface. Display 74 includes a numerical measurement 76 of the patient's cornea in microns (pm) and the waveform 78 (e.g., pulse) used to obtain the measurement. Also provided on display 74 are the speed 80 of sound 78, bias information 81, and other information 85. The bias information corresponds to a percentage (e.g., 90%) of the measured corneal thickness. The other information 85 includes an average value (e.g., “452” in FIG. 5) and a standard deviation (e.g., “0.01” in FIG. 5). In this regard, hand-held computing device 14 stores numerous measurements for a single patient. These measurements may be displayed in a list 89 (see also FIG. 6 —a scroll bar 91 is provided to scroll through the list). The average value is the average of the values on list 89. The standard. deviation is the standard deviation of the values on list 89. FIG. 6 shows a close-up of another display 82. Display 82 is similar to display 74 of FIG. 5.

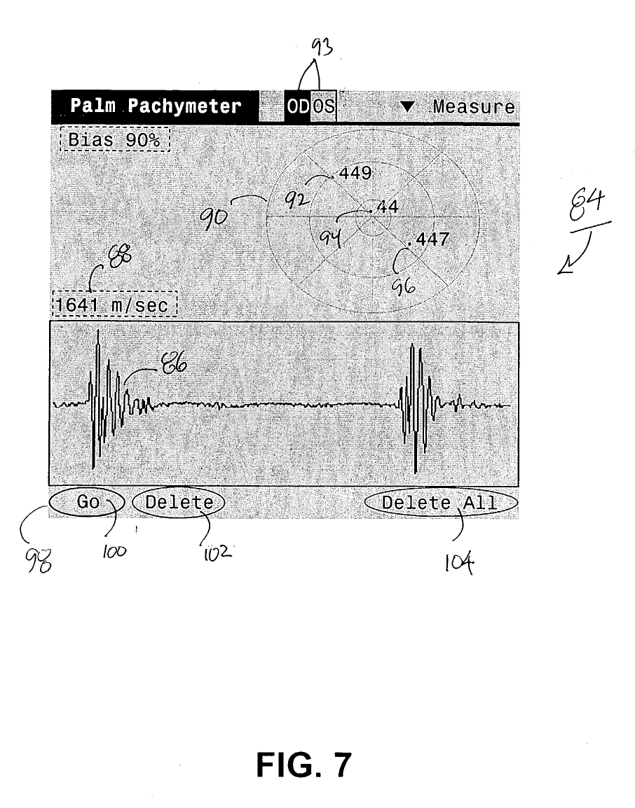

FIG. 7 shows an example of another display 84 that may be provided on hand-held computing device 14. Display 84 includes waveform 86, the speed 88 of sound, and a map 90 of the patient's eye. Map 90 includes measurements that correspond to the thickness of the cornea at points 92, 94 (center) and 96.

As shown in FIGS. 5 to 7, the display may include information indicating to which eye (left or right) the on-screen measurement information applies. “OS” indicates that the measurement information applies to the left eye and “OD” indicates that the measurement information applies to the right eye. A user may select either “OS” or “OD” using an input device, such as a touch screen stylus.

Hand-held computing device 14 may maintain a patient database in memory (not shown). This patient database may store information by patient using, e.g., a patient identifier (ID). The patient information may include measurements taken using pachymeter 10, including the average values, standard deviations, and waveforms, if desired. The patient database may be downloaded to a desktop personal computer, printed, etc. in a conventional manner from the hand-held computing device.

Audio feedback may also be provided via an audio transducer, which may be contained on hand-held computing device 14 or expansion module 18. For example, to obtain the best reading, probe 12 should be normal (orthogonal) to the surface of a patient's cornea. Audio feedback can be provided to indicate when the probe is in the proper position relative to the cornea. Also, audio feedback can be provided to indicate good corneal contact or that a good reading has been obtained. The same, or different, sounds may be used to indicate different functions. Software in expansion module 18 and hand-held computing device 14 may work in conjunction to provide this functionality.

User input, including commands and the like, may be provided through touch screen and push-button interfaces on the hand-held computing device. FIG. 7 shows an example of such a user interface 98. Go button 100 allows a user to proceed with a measurement of corneal thickness. Delete button 102 allows a user to delete information relating to a patient measurement from list 89. Delete All button 104 allows a user to delete stored information for all measurements in the list (i.e., the patient's history of measurements). Other controls for entering commands, in addition to those shown in the figure, may be provided.

As noted above, software 68 on expansion module 18 interfaces with the native operating system of hand-held computing device 14. To this end, software 68 provides functions enabling the installation, invocation, and termination of software 16 on hand-held computing device 14, in addition to functions for interfacing to standard hardware located within hand-held computing device 14.

Likewise, software 16 controls and responds to hardware contained on expansion module 18. Examples of such control and response include, but are not limited to, configuring various hardware modes, setting operational parameters, controlling sequencing, reading resultant data from the hardware, and managing power distribution.

Examples of diagnostic measuring instruments that could be implemented using this expansion module include, but are not limited to, the pachymeter described above, an ultrasonic A-scan biometer for measuring axial dimensions of an eye, an ultrasonic B-scan for obtaining two-dimensional images of eye structures, a tonometer for measuring pressure within an eye, and a keratometer for measuring the optical power and/or radius of curvature of a cornea. More than one such diagnostic measuring instrument may be implemented using a single expansion module. Alternatively, multiple expansion modules may be used to implement different diagnostic measuring instruments.

In the ultrasonic A-scan biometer embodiment, processing logic 50 measures echoes from the lens and retina in order to determine the location of the lens and the eye's axial dimensions (the measurement from the anterior of the cornea to the retina). A 10 MHz probe may be used rather than the 20 MHz probe noted above.

In the ultrasonic B-scan embodiment, the probe is placed at a point on the eye. The probe propagates ultrasonic pulses into the eye at different angles. Structures in the eye are imaged based on the strength of echoes resulting from the ultrasonic pulses. Strong echoes produce lighter (e.g., white) image areas (e.g., pixels), whereas weaker echoes produce darker (e.g., black) image areas. Intermediate-strength echoes produce gray image areas, with the strength of the echo determining the “darkness” of the gray image areas.

In the tonometer embodiment, probe 12 may be a strain gauge, pressure transducer, or force gauge that is pressed against the eye to determine eye pressure. In this case, the signals provided by probe 12 would not be ultrasonic.

In the keratometer embodiment, the probe may be optical. The probe shines optical signals (e.g., light rays) into the patient's eye. The probe receives optical reflections off of the cornea, which are used by the expansion module and computing device to determine the shape (e.g., curvature) of the cornea.

Expansion module 18 is not limited to the hardware/software configuration of FIGS. 1 and 3; it may find applicability in any computing or processing environment. Expansion module 18 may be implemented in hardware (e.g., an ASIC {Application-Specific Integrated Circuit}, dedicated logic circuits, and/or an FPGA), software, or a combination of hardware and software.

Each computer program on expansion module 18 and/or hand-held computing device 14 may be implemented in a high level procedural or object-oriented programming language to communicate with a computer system. Also, the programs can be implemented in assembly or machine language. The language may be a compiled or an interpreted language.

Each computer program may be stored on a storage medium or device that is readable by a general or special purpose processor for configuring and operating the device.

The benefits of the hand-held ophthalmic devices described herein include: ease of use due to familiar user interfaces and operating system conventions that reduce the learning curve; higher performance at a lower price due to the economies of scale realized by manufacturers of hand-held computing devices; increased miniaturization and integration; and enhanced functionality, such as infrared printing and easy synchronization with a desktop computer.

The expansion module described herein is used as an ophthalmic device. However, the expansion module and concepts described herein are not limited to use with ophthalmic devices or to use with the eye. Rather, the expansion module and concepts described herein may be used to produce any type of hand-held medical device.

The division of labor between expansion module 18 and hand-held computing device 14 may vary in different embodiments. For example, in one embodiment described above, processing logic 50 performs signal processing, whereafter a processor on hand-held computing device 14 makes determinations (e.g., corneal thickness) based on the signals processed by processing logic 50. The invention, however, is not limited as such. For example, all signal processing, including making determinations such as corneal thickness, may be performed on expansion module 18 using processing logic 50 or additional processing circuitry. Alternatively, one or more signal processing functions, such as averaging and correlating, may be performed by the processor on hand-held computing device 14.

The devices and applications described herein may all be used in veterinary medicine as well as with human subjects.

Other embodiments not described herein are also within the scope of the following claims.

What is claimed is:

1. An ultrasonic device, comprising:
a probe to receive an ultrasonic signal from an eye;
a hand-held computing device; and
an expansion module which interfaces the probe to the hand-held computing device, the expansion module comprising circuitry to process the ultrasonic signal and executable instructions to obtain a characteristic of the eye based on the ultrasonic signal.

2. The ultrasonic device of claim 1, wherein the probe comprises a piezoelectric transducer.

3. The ultrasonic device of claim 2, wherein the piezoelectric transducer generates a pulse which propagates to a structure in the eye and receives an echo of the pulse off of the structure, the echo comprising the ultrasonic signal.

4. The ultrasonic device of claim 3, wherein the structure comprises at least one of a cornea of the eye, a lens of the eye, and a retina of the eye.

5. The ultrasonic device of claim 1, wherein the hand-held computing device includes a user interface, the user interface for displaying data that corresponds to the ultrasonic signal.

6. The ultrasonic device of claim 1, wherein the hand-held computing device comprises a user interface, the user interface for inputting commands.

7. The ultrasonic device of claim 1, wherein the circuitry comprises:
a memory that stores the executable instructions;
circuits to produce a digitized ultrasonic signal from the ultrasonic signal; and
processing logic which performs processing of the digitized ultrasonic signal.

8. The ultrasonic device of claim 7, wherein processing the digitized ultrasonic signal comprises filtering the digitized ultrasonic signal to reduce noise.

9. The ultrasonic device of claim 7, wherein processing the digitized ultrasonic signal comprises combining the digitized ultrasonic signal with other digital signals to produce an averaged signal.

10. The ultrasonic device of claim 7, wherein processing the digitized ultrasonic signal comprises performing a correlation operation on the digitized ultrasonic signal.

11. The ultrasonic device of claim 7, wherein processing the digitized ultrasonic signal comprises performing peak detection to locate a feature of the digitized ultrasonic signal.

12. The ultrasonic device of claim 7, wherein processing the digitized ultrasonic signal comprises performing an interpolation using the digitized ultrasonic signal.

13. The ultrasonic device of claim 1, wherein the expansion module includes an expansion slot interface to the hand-held computing device.

14. A hand-held pachymeter comprising:
an ultrasonic probe capable of emitting ultrasonic pulses and capable of receiving echo signals of the ultrasonic pulses, the echo signals comprising an echo signal from an anterior portion of a cornea of an eye and an echo signal from a posterior portion of the cornea;
a hand-held computing device; and
an expansion module that interfaces the probe to the hand-held computing device, the expansion module comprising:
circuitry to produce digitized signals that correspond to the echo signals; and
a memory which stores computer instructions to determine a thickness of the cornea based on the digitized signals, the computer instructions being executed by the hand-held computing device.

15. The hand-held pachymeter of claim 14, wherein the ultrasonic probe comprises a piezoelectric transducer.

16. The hand-held pachymeter of claim 14, wherein the circuitry comprises:
an amplifier to amplify the echo signals; and
a digitizer to digitize amplified echo signals to produce the digitized signals.

17. The hand-held pachymeter of claim 14, wherein the circuitry filters the digitized signals to reduce noise.

18. The hand-held pachymeter of claim 14, wherein the circuitry combines the digitized signals with other digital signals to produce an averaged digitized signal.

19. The hand-held pachymeter of claim 14, wherein the circuitry performs a correlation operation on a digitized signal.

20. The hand-held pachymeter of claim 14, wherein the circuitry performs peak detection to locate a feature of a digitized signal.

21. The hand-held pachymeter of claim 14, wherein the circuitry performs an interpolation using the digitized signals.

22. A hand-held ultrasonic device, comprising:
receiving means for receiving an ultrasonic signal from a structure of an eye; and
processing means for processing the ultrasonic signal to determine a characteristic of the structure, the processing means including a hand-held computing device for receiving data from the receiving means that is used in determining the characteristic of the structure.

23. The hand-held ultrasonic device of claim 22, wherein the receiving means comprises an ultrasonic probe capable of emitting ultrasonic pulses and capable of receiving ultrasonic echo signals of the ultrasonic pulses, the ultrasonic echo signals comprising an echo signal from an anterior portion of a cornea of an eye and an echo signal from a posterior portion of the cornea.

24. The hand-held ultrasonic device of claim 23, wherein the processing means comprises:
circuitry to produce digitized signals that correspond to the ultrasonic echo signals; and
a processor to determine a thickness of the cornea based on the digitized signals.

25. The hand-held ultrasonic device of claim 23, wherein the receiving means comprises a transducer that generates a pulse that propagates to a structure in the eye and receives an echo of the pulse off of the structure, the echo comprising the ultrasonic signal.

26. The ultrasonic device of claim 25, wherein the structure comprises at least one of a cornea of the eye, a lens of the eye, and a retina of the eye.

27. A medical device, comprising:
a probe to receive a signal from an eye;
a hand-held computing device; and
an expansion module which interfaces the probe to the hand-held computing device, the expansion module comprising circuitry to process the signal and to provide a processed version of the signal to the hand-held computing device.

28. The medical device of claim 27, wherein the probe comprises an ultrasonic probe.

29. The medical device of claim 27, wherein the probe comprises at least one of a strain gauge, a pressure transducer, and a force gauge.

30. The medical device of claim 29, wherein the characteristic of the eye comprises pressure in the eye.

31. The medical device of claim 27, wherein:
the probe comprises an ultrasonic probe capable of emitting ultrasonic pulses and capable of receiving ultrasonic echo signals of the ultrasonic pulses, the signal comprising at least one of an ultrasonic echo signal from an anterior portion of a cornea of an eye and an ultrasonic echo signal from a posterior portion of the cornea; and
the circuitry comprises:
circuitry to produce digitized signals that correspond to the ultrasonic echo signals; and
processing logic to determine a thickness of the cornea based on the digitized signals.
